SEQUENCE LISTING (247 sequences)

SEQ ID NO: 1 - DNA, 49 bp, synthetic construct
  source 1..49: mol_type = other DNA
  variation 39..48: note = Ns at the positions 39 to 48 represent the forward sequence of the Index tag, with a length ranging from 4 to 10 bases.
ttgtcttcctaaggaacgacatggctacgatccgacttnnnnnnnnnnt

SEQ ID NO: 2 - DNA, 59 bp, synthetic construct
  source 1..59: mol_type = other DNA
  variation 1..10: note = Ns at the positions 1 to 10 represent the reverse complementary sequence of the Index tag, with a length ranging from 4 to 10 bases.
nnnnnnnnnnaagtcggaggccaagcggtcttaggaagacaactcacgagtaggctaga

SEQ ID NO: 3 - DNA, 17 bp, synthetic construct
  source 1..17: mol_type = other DNA
gaacgacatggctacga

SEQ ID NO: 4 - DNA, 44 bp, synthetic construct
  source 1..44: mol_type = other DNA
  variation 18..27: note = Ns at the positions 18 to 27 represent the sequence of Barcode tag.
tgtgagccaaggagttgnnnnnnnnnntctagcctactcgtgag

SEQ ID NO: 5 - DNA, 24 bp, synthetic construct
  source 1..24: mol_type = other DNA
gccatgtcgttctgtgagccaagg

SEQ ID NO: 6-46 - skipped (41 entries)

SEQ ID NO: 47 - DNA, 10 bp, synthetic construct
  source 1..10: mol_type = other DNA
ctacacggaa

SEQ ID NO: 48 - DNA, 10 bp, synthetic construct
  source 1..10: mol_type = other DNA
tctggcaacg

SEQ ID NO: 49 - DNA, 10 bp, synthetic construct
  source 1..10: mol_type = other DNA
gagacgacca

SEQ ID NO: 50-53 - skipped (4 entries)

SEQ ID NO: 54 - DNA, 10 bp, synthetic construct
  source 1..10: mol_type = other DNA
gtgagtgatg

SEQ ID NO: 55 - DNA, 10 bp, synthetic construct
  source 1..10: mol_type = other DNA
gagtcagctg

SEQ ID NO: 56 - DNA, 10 bp, synthetic construct
  source 1..10: mol_type = other DNA
tgtctgcgaa

SEQ ID NO: 57 - DNA, 10 bp, synthetic construct
  source 1..10: mol_type = other DNA
attggtacaa

SEQ ID NO: 58 - DNA, 10 bp, synthetic construct
  source 1..10: mol_type = other DNA
cgattgtggt

SEQ ID NO: 59 - DNA, 10 bp, synthetic construct
  source 1..10: mol_type = other DNA
acagacttcc

SEQ ID NO: 60 - DNA, 10 bp, synthetic construct
  source 1..10: mol_type = other DNA
tccacactct

SEQ ID NO: 61 - DNA, 10 bp, synthetic construct
  source 1..10: mol_type = other DNA
caccacaagc

SEQ ID NO: 62 - DNA, 10 bp, synthetic construct
  source 1..10: mol_type = other DNA
tagaggacaa

SEQ ID NO: 63 - DNA, 10 bp, synthetic construct
  source 1..10: mol_type = other DNA
cctagcgaat

SEQ ID NO: 64 - DNA, 10 bp, synthetic construct
  source 1..10: mol_type = other DNA
gtagtcatcg

SEQ ID NO: 65 - DNA, 10 bp, synthetic construct
  source 1..10: mol_type = other DNA
gctgagctgt

SEQ ID NO: 66 - DNA, 10 bp, synthetic construct
  source 1..10: mol_type = other DNA
aacctagata

SEQ ID NO: 67 - DNA, 10 bp, synthetic construct
  source 1..10: mol_type = other DNA
ttgccatctc

SEQ ID NO: 68 - DNA, 10 bp, synthetic construct
  source 1..10: mol_type = other DNA
agatcttgcg

SEQ ID NO: 69 - DNA, 10 bp, synthetic construct
  source 1..10: mol_type = other DNA
cgctatcggc

SEQ ID NO: 70 - DNA, 10 bp, synthetic construct
  source 1..10: mol_type = other DNA
gcaacgatgg

SEQ ID NO: 71 - DNA, 10 bp, synthetic construct
  source 1..10: mol_type = other DNA
taatcgttca

SEQ ID NO: 72 - DNA, 10 bp, synthetic construct
  source 1..10: mol_type = other DNA
gttcgctcta

SEQ ID NO: 73 - DNA, 10 bp, synthetic construct
  source 1..10: mol_type = other DNA
tctcacacat

SEQ ID NO: 74 - DNA, 10 bp, synthetic construct
  source 1..10: mol_type = other DNA
ctgttaggat

SEQ ID NO: 75 - DNA, 10 bp, synthetic construct
  source 1..10: mol_type = other DNA
cgcagacgcg

SEQ ID NO: 76 - DNA, 10 bp, synthetic construct
  source 1..10: mol_type = other DNA
aaggatcatc

SEQ ID NO: 77 - DNA, 10 bp, synthetic construct
  source 1..10: mol_type = other DNA
agcgttgagc

SEQ ID NO: 78 - DNA, 10 bp, synthetic construct
  source 1..10: mol_type = other DNA
ttagatgcat

SEQ ID NO: 79 - DNA, 10 bp, synthetic construct
  source 1..10: mol_type = other DNA
gtccagagct

SEQ ID NO: 80 - DNA, 10 bp, synthetic construct
  source 1..10: mol_type = other DNA
cacgtgatag

SEQ ID NO: 81 - DNA, 10 bp, synthetic construct
  source 1..10: mol_type = other DNA
ccactagtcc

SEQ ID NO: 82 - DNA, 10 bp, synthetic construct
  source 1..10: mol_type = other DNA
tggacttggc

SEQ ID NO: 83 - DNA, 10 bp, synthetic construct
  source 1..10: mol_type = other DNA
gcttgacagg

SEQ ID NO: 84 - DNA, 10 bp, synthetic construct
  source 1..10: mol_type = other DNA
aagacctcta

SEQ ID NO: 85 - DNA, 10 bp, synthetic construct
  source 1..10: mol_type = other DNA
agttgccata

SEQ ID NO: 86 - DNA, 10 bp, synthetic construct
  source 1..10: mol_type = other DNA
atgtacgcag

SEQ ID NO: 87 - DNA, 10 bp, synthetic construct
  source 1..10: mol_type = other DNA
ttaatgagat

SEQ ID NO: 88 - DNA, 10 bp, synthetic construct
  source 1..10: mol_type = other DNA
tgcgccactt

SEQ ID NO: 89 - DNA, 10 bp, synthetic construct
  source 1..10: mol_type = other DNA
cattaaggcc

SEQ ID NO: 90 - DNA, 10 bp, synthetic construct
  source 1..10: mol_type = other DNA
ccgcctcaga

SEQ ID NO: 91 - DNA, 10 bp, synthetic construct
  source 1..10: mol_type = other DNA
aatcggctcg

SEQ ID NO: 92 - DNA, 10 bp, synthetic construct
  source 1..10: mol_type = other DNA
gccggttatc

SEQ ID NO: 93 - DNA, 10 bp, synthetic construct
  source 1..10: mol_type = other DNA
ggaatattga

SEQ ID NO: 94 - DNA, 10 bp, synthetic construct
  source 1..10: mol_type = other DNA
attcaacgga

SEQ ID NO: 95 - DNA, 10 bp, synthetic construct
  source 1..10: mol_type = other DNA
aactgtactg

SEQ ID NO: 96 - DNA, 10 bp, synthetic construct
  source 1..10: mol_type = other DNA
gtacctcaat

SEQ ID NO: 97 - DNA, 10 bp, synthetic construct
  source 1..10: mol_type = other DNA
gacttctaat

SEQ ID NO: 98 - DNA, 10 bp, synthetic construct
  source 1..10: mol_type = other DNA
tgaagcgttg

SEQ ID NO: 99 - DNA, 10 bp, synthetic construct
  source 1..10: mol_type = other DNA
cgtgcgatcc

SEQ ID NO: 100 - DNA, 10 bp, synthetic construct
  source 1..10: mol_type = other DNA
tcggaaggca

SEQ ID NO: 101 - DNA, 10 bp, synthetic construct
  source 1..10: mol_type = other DNA
ccgatgtcgc

SEQ ID NO: 102 - DNA, 10 bp, synthetic construct
  source 1..10: mol_type = other DNA
acttagaatg

SEQ ID NO: 103 - DNA, 10 bp, synthetic construct
  source 1..10: mol_type = other DNA
tccaagcctg

SEQ ID NO: 104 - DNA, 10 bp, synthetic construct
  source 1..10: mol_type = other DNA
agacgatgat

SEQ ID NO: 105 - DNA, 10 bp, synthetic construct
  source 1..10: mol_type = other DNA
ctcacaagac

SEQ ID NO: 106 - DNA, 10 bp, synthetic construct
  source 1..10: mol_type = other DNA
cgttcctact

SEQ ID NO: 107 - DNA, 10 bp, synthetic construct
  source 1..10: mol_type = other DNA
gtggttgtga

SEQ ID NO: 108 - DNA, 10 bp, synthetic construct
  source 1..10: mol_type = other DNA
gaaggcctgc

SEQ ID NO: 109 - DNA, 10 bp, synthetic construct
  source 1..10: mol_type = other DNA
tagcttgcca

SEQ ID NO: 110 - DNA, 10 bp, synthetic construct
  source 1..10: mol_type = other DNA
gacaatgctc

SEQ ID NO: 111 - DNA, 10 bp, synthetic construct
  source 1..10: mol_type = other DNA
gctaatcaca

SEQ ID NO: 112 - DNA, 10 bp, synthetic construct
  source 1..10: mol_type = other DNA
agtccatagg

SEQ ID NO: 113 - DNA, 10 bp, synthetic construct
  source 1..10: mol_type = other DNA
ctatcgccta

SEQ ID NO: 114 - DNA, 10 bp, synthetic construct
  source 1..10: mol_type = other DNA
atcgtggtct

SEQ ID NO: 115 - DNA, 10 bp, synthetic construct
  source 1..10: mol_type = other DNA
tggctaatac

SEQ ID NO: 116 - DNA, 10 bp, synthetic construct
  source 1..10: mol_type = other DNA
cagtgcagag

SEQ ID NO: 117 - DNA, 10 bp, synthetic construct
  source 1..10: mol_type = other DNA
tcaggctggt

SEQ ID NO: 118 - DNA, 10 bp, synthetic construct
  source 1..10: mol_type = other DNA
atactcacgc

SEQ ID NO: 119 - DNA, 10 bp, synthetic construct
  source 1..10: mol_type = other DNA
atgctccgcg

SEQ ID NO: 120 - DNA, 10 bp, synthetic construct
  source 1..10: mol_type = other DNA
tgtgaacttg

SEQ ID NO: 121 - DNA, 10 bp, synthetic construct
  source 1..10: mol_type = other DNA
gagaggtgct

SEQ ID NO: 122 - DNA, 10 bp, synthetic construct
  source 1..10: mol_type = other DNA
tgcactgtaa

SEQ ID NO: 123 - DNA, 10 bp, synthetic construct
  source 1..10: mol_type = other DNA
gcctaggcaa

SEQ ID NO: 124 - DNA, 10 bp, synthetic construct
  source 1..10: mol_type = other DNA
ccatcatagc

SEQ ID NO: 125 - DNA, 10 bp, synthetic construct
  source 1..10: mol_type = other DNA
catggtaatt

SEQ ID NO: 126 - DNA, 10 bp, synthetic construct
  source 1..10: mol_type = other DNA
caccatgtct

SEQ ID NO: 127 - DNA, 10 bp, synthetic construct
  source 1..10: mol_type = other DNA
atatgtctgg

SEQ ID NO: 128 - DNA, 10 bp, synthetic construct
  source 1..10: mol_type = other DNA
aaggaagcgt

SEQ ID NO: 129 - DNA, 10 bp, synthetic construct
  source 1..10: mol_type = other DNA
tcaagacgtc

SEQ ID NO: 130 - DNA, 10 bp, synthetic construct
  source 1..10: mol_type = other DNA
ccgctcagta

SEQ ID NO: 131 - DNA, 10 bp, synthetic construct
  source 1..10: mol_type = other DNA
ggtgtgtaca

SEQ ID NO: 132 - DNA, 10 bp, synthetic construct
  source 1..10: mol_type = other DNA
ttcacgtaag

SEQ ID NO: 133 - DNA, 10 bp, synthetic construct
  source 1..10: mol_type = other DNA
ggttccacac

SEQ ID NO: 134 - DNA, 10 bp, synthetic construct
  source 1..10: mol_type = other DNA
agctgcgcac

SEQ ID NO: 135 - DNA, 10 bp, synthetic construct
  source 1..10: mol_type = other DNA
gattaatgaa

SEQ ID NO: 136 - DNA, 10 bp, synthetic construct
  source 1..10: mol_type = other DNA
ccaatgcttg

SEQ ID NO: 137 - DNA, 10 bp, synthetic construct
  source 1..10: mol_type = other DNA
ttgcctatgt

SEQ ID NO: 138 - DNA, 10 bp, synthetic construct
  source 1..10: mol_type = other DNA
aacgctcacg

SEQ ID NO: 139 - DNA, 10 bp, synthetic construct
  source 1..10: mol_type = other DNA
ccgatcggcc

SEQ ID NO: 140 - DNA, 10 bp, synthetic construct
  source 1..10: mol_type = other DNA
ttaggaacta

SEQ ID NO: 141 - DNA, 10 bp, synthetic construct
  source 1..10: mol_type = other DNA
ggtcagtagt

SEQ ID NO: 142 - DNA, 10 bp, synthetic construct
  source 1..10: mol_type = other DNA
tgccggatca

SEQ ID NO: 143 - DNA, 10 bp, synthetic construct
  source 1..10: mol_type = other DNA
actcatgacc

SEQ ID NO: 144 - DNA, 10 bp, synthetic construct
  source 1..10: mol_type = other DNA
caagtctcat

SEQ ID NO: 145 - DNA, 10 bp, synthetic construct
  source 1..10: mol_type = other DNA
gtgacatgtg

SEQ ID NO: 146 - DNA, 10 bp, synthetic construct
  source 1..10: mol_type = other DNA
aagtacctgg

SEQ ID NO: 147 - DNA, 10 bp, synthetic construct
  source 1..10: mol_type = other DNA
cctatagcac

SEQ ID NO: 148 - DNA, 10 bp, synthetic construct
  source 1..10: mol_type = other DNA
ttcggtcagt

SEQ ID NO: 149 - DNA, 10 bp, synthetic construct
  source 1..10: mol_type = other DNA
ggatcgagta

SEQ ID NO: 150 - DNA, 10 bp, synthetic construct
  source 1..10: mol_type = other DNA
aggacgtaga

SEQ ID NO: 151 - DNA, 10 bp, synthetic construct
  source 1..10: mol_type = other DNA
ctgaaccgaa

SEQ ID NO: 152 - DNA, 10 bp, synthetic construct
  source 1..10: mol_type = other DNA
gaacgtgtcg

SEQ ID NO: 153 - DNA, 10 bp, synthetic construct
  source 1..10: mol_type = other DNA
tccgtgactc

SEQ ID NO: 154 - DNA, 10 bp, synthetic construct
  source 1..10: mol_type = other DNA
aattcactgt

SEQ ID NO: 155 - DNA, 10 bp, synthetic construct
  source 1..10: mol_type = other DNA
cctgaaggat

SEQ ID NO: 156 - DNA, 10 bp, synthetic construct
  source 1..10: mol_type = other DNA
ttccttactg

SEQ ID NO: 157 - DNA, 10 bp, synthetic construct
  source 1..10: mol_type = other DNA
ggatgctacc

SEQ ID NO: 158 - DNA, 10 bp, synthetic construct
  source 1..10: mol_type = other DNA
cgacgatatg

SEQ ID NO: 159 - DNA, 10 bp, synthetic construct
  source 1..10: mol_type = other DNA
gacggtcgag

SEQ ID NO: 160 - DNA, 10 bp, synthetic construct
  source 1..10: mol_type = other DNA
acgaaggtcc

SEQ ID NO: 161 - DNA, 10 bp, synthetic construct
  source 1..10: mol_type = other DNA
ttaacgacga

SEQ ID NO: 162 - DNA, 10 bp, synthetic construct
  source 1..10: mol_type = other DNA
aattccgagt

SEQ ID NO: 163 - DNA, 10 bp, synthetic construct
  source 1..10: mol_type = other DNA
cctgtaacca

SEQ ID NO: 164 - DNA, 10 bp, synthetic construct
  source 1..10: mol_type = other DNA
ttgcttcgtt

SEQ ID NO: 165 - DNA, 10 bp, synthetic construct
  source 1..10: mol_type = other DNA
ggctacttac

SEQ ID NO: 166 - DNA, 10 bp, synthetic construct
  source 1..10: mol_type = other DNA
taatggcctt

SEQ ID NO: 167 - DNA, 10 bp, synthetic construct
  source 1..10: mol_type = other DNA
gtgaccggta

SEQ ID NO: 168 - DNA, 10 bp, synthetic construct
  source 1..10: mol_type = other DNA
accgattagg

SEQ ID NO: 169 - DNA, 10 bp, synthetic construct
  source 1..10: mol_type = other DNA
cgtctgatat

SEQ ID NO: 170 - DNA, 10 bp, synthetic construct
  source 1..10: mol_type = other DNA
aacgcattcc

SEQ ID NO: 171 - DNA, 10 bp, synthetic construct
  source 1..10: mol_type = other DNA
ccgagagacc

SEQ ID NO: 172 - DNA, 10 bp, synthetic construct
  source 1..10: mol_type = other DNA
tgattccgag

SEQ ID NO: 173 - DNA, 10 bp, synthetic construct
  source 1..10: mol_type = other DNA
gttcatacga

SEQ ID NO: 174 - DNA, 10 bp, synthetic construct
  source 1..10: mol_type = other DNA
tagactgatc

SEQ ID NO: 175 - DNA, 10 bp, synthetic construct
  source 1..10: mol_type = other DNA
gattccatga

SEQ ID NO: 176 - DNA, 10 bp, synthetic construct
  source 1..10: mol_type = other DNA
acacagtgct

SEQ ID NO: 177 - DNA, 10 bp, synthetic construct
  source 1..10: mol_type = other DNA
cgcgtaccag

SEQ ID NO: 178 - DNA, 10 bp, synthetic construct
  source 1..10: mol_type = other DNA
atacgctaga

SEQ ID NO: 179 - DNA, 10 bp, synthetic construct
  source 1..10: mol_type = other DNA
ccggatactc

SEQ ID NO: 180 - DNA, 10 bp, synthetic construct
  source 1..10: mol_type = other DNA
tgcttgcgcg

SEQ ID NO: 181 - DNA, 10 bp, synthetic construct
  source 1..10: mol_type = other DNA
gttagagtat

SEQ ID NO: 182 - DNA, 10 bp, synthetic construct
  source 1..10: mol_type = other DNA
tgctatgcac

SEQ ID NO: 183 - DNA, 10 bp, synthetic construct
  source 1..10: mol_type = other DNA
gtgcatagag

SEQ ID NO: 184 - DNA, 10 bp, synthetic construct
  source 1..10: mol_type = other DNA
aatgcgtact

SEQ ID NO: 185 - DNA, 10 bp, synthetic construct
  source 1..10: mol_type = other DNA
ccaggaccta

SEQ ID NO: 186 - DNA, 10 bp, synthetic construct
  source 1..10: mol_type = other DNA
aagctcatga

SEQ ID NO: 187 - DNA, 10 bp, synthetic construct
  source 1..10: mol_type = other DNA
cctagagttg

SEQ ID NO: 188 - DNA, 10 bp, synthetic construct
  source 1..10: mol_type = other DNA
tgcatccggt

SEQ ID NO: 189 - DNA, 10 bp, synthetic construct
  source 1..10: mol_type = other DNA
gtatcgtacc

SEQ ID NO: 190 - DNA, 10 bp, synthetic construct
  source 1..10: mol_type = other DNA
gtatcgaatt

SEQ ID NO: 191 - DNA, 10 bp, synthetic construct
  source 1..10: mol_type = other DNA
cgtactacgt

SEQ ID NO: 192 - DNA, 10 bp, synthetic construct
  source 1..10: mol_type = other DNA
aacgtactcg

SEQ ID NO: 193 - DNA, 10 bp, synthetic construct
  source 1..10: mol_type = other DNA
tcgcactgac

SEQ ID NO: 194 - DNA, 10 bp, synthetic construct
  source 1..10: mol_type = other DNA
aactgtgtga

SEQ ID NO: 195 - DNA, 10 bp, synthetic construct
  source 1..10: mol_type = other DNA
ccgatgcaca

SEQ ID NO: 196 - DNA, 10 bp, synthetic construct
  source 1..10: mol_type = other DNA
tgtgacgcag

SEQ ID NO: 197 - DNA, 10 bp, synthetic construct
  source 1..10: mol_type = other DNA
gtacgatgtc

SEQ ID NO: 198 - DNA, 10 bp, synthetic construct
  source 1..10: mol_type = other DNA
ctcgttccgg

SEQ ID NO: 199 - DNA, 10 bp, synthetic construct
  source 1..10: mol_type = other DNA
cgtgccagga

SEQ ID NO: 200 - DNA, 10 bp, synthetic construct
  source 1..10: mol_type = other DNA
aagcgatatc

SEQ ID NO: 201 - DNA, 10 bp, synthetic construct
  source 1..10: mol_type = other DNA
gcaactgtct

SEQ ID NO: 202 - DNA, 10 bp, synthetic construct
  source 1..10: mol_type = other DNA
taatggtcag

SEQ ID NO: 203 - DNA, 10 bp, synthetic construct
  source 1..10: mol_type = other DNA
acctaggacc

SEQ ID NO: 204 - DNA, 10 bp, synthetic construct
  source 1..10: mol_type = other DNA
ttgcacatta

SEQ ID NO: 205 - DNA, 10 bp, synthetic construct
  source 1..10: mol_type = other DNA
ggtatacgat

SEQ ID NO: 206 - DNA, 10 bp, synthetic construct
  source 1..10: mol_type = other DNA
cacacacagg

SEQ ID NO: 207 - DNA, 10 bp, synthetic construct
  source 1..10: mol_type = other DNA
tagtgctgtc

SEQ ID NO: 208 - DNA, 10 bp, synthetic construct
  source 1..10: mol_type = other DNA
acactgatat

SEQ ID NO: 209 - DNA, 10 bp, synthetic construct
  source 1..10: mol_type = other DNA
ggtaatgcct

SEQ ID NO: 210 - DNA, 10 bp, synthetic construct
  source 1..10: mol_type = other DNA
atagcacaga

SEQ ID NO: 211 - DNA, 10 bp, synthetic construct
  source 1..10: mol_type = other DNA
cctgacatcg

SEQ ID NO: 212 - DNA, 10 bp, synthetic construct
  source 1..10: mol_type = other DNA
ttgctgtgac

SEQ ID NO: 213 - DNA, 10 bp, synthetic construct
  source 1..10: mol_type = other DNA
ggctgtgcta

SEQ ID NO: 214 - DNA, 10 bp, synthetic construct
  source 1..10: mol_type = other DNA
aggcactgcc

SEQ ID NO: 215 - DNA, 10 bp, synthetic construct
  source 1..10: mol_type = other DNA
cgtcgttaac

SEQ ID NO: 216 - DNA, 10 bp, synthetic construct
  source 1..10: mol_type = other DNA
gaagtaatgt

SEQ ID NO: 217 - DNA, 10 bp, synthetic construct
  source 1..10: mol_type = other DNA
tccagtgcta

SEQ ID NO: 218 - DNA, 10 bp, synthetic construct
  source 1..10: mol_type = other DNA
atatcgcaag

SEQ ID NO: 219 - DNA, 10 bp, synthetic construct
  source 1..10: mol_type = other DNA
cacgcagttg

SEQ ID NO: 220 - DNA, 10 bp, synthetic construct
  source 1..10: mol_type = other DNA
ttgatccgga

SEQ ID NO: 221 - DNA, 10 bp, synthetic construct
  source 1..10: mol_type = other DNA
gcttagacct

SEQ ID NO: 222 - DNA, 10 bp, synthetic construct
  source 1..10: mol_type = other DNA
gacagattgg

SEQ ID NO: 223 - DNA, 10 bp, synthetic construct
  source 1..10: mol_type = other DNA
gaattggccg

SEQ ID NO: 224 - DNA, 10 bp, synthetic construct
  source 1..10: mol_type = other DNA
acgcatcaat

SEQ ID NO: 225 - DNA, 10 bp, synthetic construct
  source 1..10: mol_type = other DNA
cgtgacagtc

SEQ ID NO: 226 - DNA, 10 bp, synthetic construct
  source 1..10: mol_type = other DNA
ttaacttgaa

SEQ ID NO: 227 - DNA, 10 bp, synthetic construct
  source 1..10: mol_type = other DNA
acgcgaccgt

SEQ ID NO: 228 - DNA, 10 bp, synthetic construct
  source 1..10: mol_type = other DNA
tgtgtcatca

SEQ ID NO: 229 - DNA, 10 bp, synthetic construct
  source 1..10: mol_type = other DNA
ctctcggatc

SEQ ID NO: 230 - DNA, 10 bp, synthetic construct
  source 1..10: mol_type = other DNA
agtaagtgct

SEQ ID NO: 231 - DNA, 10 bp, synthetic construct
  source 1..10: mol_type = other DNA
tatactgcgt

SEQ ID NO: 232 - DNA, 10 bp, synthetic construct
  source 1..10: mol_type = other DNA
ccagtccttc

SEQ ID NO: 233 - DNA, 10 bp, synthetic construct
  source 1..10: mol_type = other DNA
gtccgtaagg

SEQ ID NO: 234 - DNA, 10 bp, synthetic construct
  source 1..10: mol_type = other DNA
aagtcattaa

SEQ ID NO: 235 - DNA, 10 bp, synthetic construct
  source 1..10: mol_type = other DNA
ccgttggatc

SEQ ID NO: 236 - DNA, 10 bp, synthetic construct
  source 1..10: mol_type = other DNA
ttcggacgca

SEQ ID NO: 237 - DNA, 10 bp, synthetic construct
  source 1..10: mol_type = other DNA
ggacacacag

SEQ ID NO: 238 - DNA, 10 bp, synthetic construct
  source 1..10: mol_type = other DNA
agctctgtgt

SEQ ID NO: 239 - DNA, 10 bp, synthetic construct
  source 1..10: mol_type = other DNA
ctgacgttac

SEQ ID NO: 240 - DNA, 10 bp, synthetic construct
  source 1..10: mol_type = other DNA
gaacgcagtg

SEQ ID NO: 241 - DNA, 10 bp, synthetic construct
  source 1..10: mol_type = other DNA
tctgaaccga

SEQ ID NO: 242 - DNA, 10 bp, synthetic construct
  source 1..10: mol_type = other DNA
aagctgaacc

SEQ ID NO: 243 - DNA, 10 bp, synthetic construct
  source 1..10: mol_type = other DNA
cctatataca

SEQ ID NO: 244 - DNA, 10 bp, synthetic construct
  source 1..10: mol_type = other DNA
ttcggtcgtg

SEQ ID NO: 245 - DNA, 10 bp, synthetic construct
  source 1..10: mol_type = other DNA
ggatacgcat

SEQ ID NO: 246 - DNA, 141 bp, synthetic construct
  source 1..141: mol_type = other DNA
  variation 26..35: note = Ns at the positions 26 to 35 represent the forward sequence of the Index tag, with a length ranging from 4 to 10 bases.
  variation 37..54: note = Ns at the positions 37 to 54 represent insert fragments.
  variation 115..124: note = Ns at the positions 115 to 124 represent the reverse complementary sequence of Barcode tag.
  variation 56..65: note = Ns at the positions 56 to 65 represent the reverse complementary sequence of the Index tag, with a length ranging from 4 to 10 bases.
gaacgacatggctacgatccgacttnnnnnnnnnntnnnnnnnnnnnnnnnnnnannnnnnnnnnaagtcggaggccaagcggtcttaggaagacaactcacgagtaggctagannnnnnnnnncaactccttggctcaca

SEQ ID NO: 247 - DNA, 141 bp, synthetic construct
  source 1..141: mol_type = other DNA
  variation 77..86: note = Ns at the positions 77 to 86 represent the forward sequence of the Index tag, with a length ranging from 4 to 10 bases.
  variation 18..27: note = Ns at the positions 18 to 27 represent the sequence of Barcode tag.
  variation 107..116: note = Ns at the positions 107 to 116 represent the the reverse complementary sequence of the Index tag, with a length ranging from 4 to 10 bases.
  variation 88..105: note = Ns at the positions 88 to 105 represent insert fragments.
tgtgagccaaggagttgnnnnnnnnnntctagcctactcgtgagttgtcttcctaagaccgcttggcctccgacttnnnnnnnnnntnnnnnnnnnnnnnnnnnnannnnnnnnnnaagtcggatcgtagccatgtcgttc

(19) United States
(12) Patent Application Publication
(10) Pub. No.: US 2026/0357243 A1
(43) Pub. Date: Nov. 12, 2026

(54) Adapter for Multi-Sample Pooling Library Construction, Kit, and Library Construction Method

(71) Applicant: HIGENTEC CO., LTD., Changsha (CN)

(72) Inventors: Gang Huang, Changsha (CN); Xiangyi Xiao, Changsha (CN); Xiangyu Wen, Changsha (CN); Zujun Huang, Changsha (CN); Meijuan Xu, Changsha (CN); Junling Wang, Changsha (CN); Bingchuan Tian, Changsha (CN); Shunxue Tang, Changsha (CN)

(21) Appl. No.: 19/150,926

(22) PCT Filed: May 30, 2023
(86) PCT No.: PCT/CN2023/097183

§ 371 Date: Jul. 25, 2025

(30) Foreign Application Priority Data
Feb. 23, 2023 (CN) 202310159997.4

(51) Int. Cl. C12N 15/10; C12Q 1/6806; C12Q 1/6855; C12Q 1/686

(52) U.S. Cl. CPC C12N 15/1065; C12N 15/1013; C12Q 1/6806; C12Q 1/6855; C12Q 1/686

(57) ABSTRACT

An adapter for multi-sample pooling library construction, a kit, and a library construction method are disclosed. The adapter comprises an adapter sense strand having a sequence as shown in SEQ ID NO: 1, and an adapter anti-sense strand having a sequence as shown in SEQ ID NO: 2; and the adapter sense strand and the adapter anti-sense strand form a double-stranded adapter after annealing. Two-level tags are designed for adapters, so that the number of available adapters is greatly increased while effectively reducing the adapter synthesis cost, so as to provide enough adapters for large-scale sample pooling sequencing, thereby effectively solving the problem of insufficient adapters frequently occurring during multi-sample pooling sequencing. Moreover, unequal-length adapters having a 4-10 bp inner-layer tag sequence can be used for construction of next-generation sequencing libraries such as common PCR product sequencing, multiplex PCR product sequencing, gene-edited progeny large-scale sequencing identification, and low-coverage whole-genome sequencing.

### CROSS-REFERENCE TO RELATED APPLICATIONS

This application is the United States national phase of International Patent Application No. PCT/CN2023/097183 filed May 30, 2023, and claims priority to Chinese Patent Application No. 202310159997.4 filed Feb. 23, 2023, the disclosures of which are hereby incorporated by reference in their entireties.

### INCORPORATION BY REFERENCE

This application includes a sequence listing in extensible Markup Language (a “xml” file) that is submitted herewith named P25CS1NW00010US-sequencelist.xml created on Jul. 24, 2025, and 191,343 bytes in size. This sequence listing is incorporated by reference herein.

### BACKGROUND

### Technical Field

The present disclosure belongs to the technical field of high-throughput sequencing, and in particular, relates to an adapter for multi-sample pooling library construction, a kit, and a library construction method

### TECHNICAL CONSIDERATIONS

High-throughput sequencing technology has become one of the most important analysis methods for molecular biology in the field of life science, which not only provides important data for basic research of life science such as genome genetic information decoding, gene expression regulation and species genetic evolution, but also plays an increasingly important role in many fields or scenarios including identification and molecular breeding of plant and animal germplasm resources, gene diagnosis and gene therapy of human diseases, identification and detection of infectious bacteria or viruses, environmental detection and treatment, etc.

There are related technologies for high-throughput sequencing based on MGI sequencing platform, which use fragmented DNA fragments, and involve connecting library construction adapters to two ends of the fragmented DNA fragments using bubble-shaped adapters; mixing every 8 connection products in an equal volume, and then performing double selection by magnetic beads to select the target fragments within the range for the next step of library construction; performing low-cycle-number PCR amplification on the product after double selection by magnetic beads to form a sequencing library structure with sequencing primer binding sequences and cyclization primer binding sequences flanking the target fragments; mixing the PCR products in equal amount for cyclization reaction; and subjecting the cyclization product to digestion by exonuclease ExoI and ExoIII to remove uncyclized, single-stranded and double-stranded DNAs, thus completing the preparation of cyclization library. In such technologies, the library construction adapter includes a Barcode tag sequence of 10-base length. MGI platform supplier provides 192 Barcode tags for use. After being connected with the adapter, each sample thus carries a Barcode tag with unique sequence. Upon sequencing the library, the sequencing data of the corresponding sample can be extracted from each sequencing lane by identifying the Barcode tag sequence. Sequencing data of 1-96 samples can be identified in one sequencing lane. However, the related technologies only design Barcode tags for the library construction adapter, with the total number of 96, 128, or 192, and the sequencing data of only 192 samples at most can be distinguished in one sequencing lane, which fails to meet the demand for loading further more samples into a lane or a chip for synchronous sequencing. Based on the library construction adapter of the related platform technology, the library can be only constructed for the DNA fragments with different bases in the same position after fragmentation. If the gene sequences of the DNA fragments to be constructed into library are identical in a certain region, it would lead to the need to increase dark reaction in the sequencing process and increase the sequencing cost, or directly lead to an error report by the sequencer and sequencing failure. The types of such DNA fragments include, but are not limited to, products amplified with the same PCR primer, DNA fragments produced by restriction endonucleases, etc.

### SUMMARY

The present disclosure aims to solve at least one of the technical problems existing in the above-mentioned related technologies. In view of this, the present disclosure provides an adapter for multi-sample pooling library construction.

The present disclosure further provides a kit for library construction based on MGI platform.

The present disclosure further provides a method for next-generation sequencing library construction.

According to a first aspect of the present disclosure, an adapter for multi-sample pooling library construction is provided, which comprises an adapter sense strand and an adapter anti-sense strand, wherein the adapter sense strand has a sequence as shown in SEQ ID NO: 1, and the adapter anti-sense strand has a sequence as shown in SEQ ID NO: 2; and the adapter sense strand and the adapter anti-sense strand form a double-stranded adapter after annealing.

In some embodiments of the present disclosure, Ns at positions 39 to 48 of the sequence of the adapter sense strand represent a forward sequence of an Index tag, with a length of 4-10 bases.

In some embodiments of the present disclosure, Ns at positions 1 to 10 of the sequence of the adapter anti-sense strand represent a reverse complementary sequence of the Index tag, with a length of 4-10 bases.

In some embodiments of the present disclosure, the Index tag includes a sequence selected from at least one of the group consisting of SEQ ID NO: 6 to SEQ ID NO: 53.

According to a second aspect of the present disclosure, a kit for library construction based on MGI platform is provided, which comprises the above-described adapter.

In some embodiments of the present disclosure, the kit further comprises a common primer, and the common primer has a sequence as shown in SEQ ID NO: 3.

In some embodiments of the present disclosure, the kit further comprises a Barcode Primer, and the Barcode Primer has a sequence as shown in SEQ ID NO: 4.

In some embodiments of the present disclosure, Ns at positions 18 to 27 of the sequence of the Barcode Primer as shown in SEQ ID NO: 4 represent a sequence of a Barcode tag. Specifically, the Barcode tag includes but is not limited to 192 Barcode tags provided by a MGI platform supplier. Specifically, sequences of the 192 Barcode tags are as shown in SEQ ID NO: 54 to SEQ ID NO: 245, respectively.

In some embodiments of the present disclosure, the kit further comprises a Splint Oligo, and he Splint Oligo has a sequence as shown in SEQ ID NO: 5.

According to a third aspect of the present disclosure, a method for next-generation sequencing library construction is provided, which comprises the following steps: connecting the adapter to a DNA fragment to obtain a DNA fragment with the adapter; and performing PCR amplification on the DNA fragment with the adapter to obtain a sequencing library.

In some embodiments of the present disclosure, the DNA fragment is provided by ultrasonic interruption, fragmentation enzyme digestion, or restriction enzyme digestion.

In some embodiments of the present disclosure, before connecting the adapter to the DNA fragment, the method further comprises a step of annealing two sequences of the adapter.

In some embodiments of the present disclosure, in the step of annealing, a mass ratio of the two sequences is 1:1.

In some embodiments of the present disclosure, the annealing is performed by incubating at 92-98 °C for 2-5 min, and cooling by 0.8-1.2 °C per 85-95 sec until 20-28 °C.

In some embodiments of the present disclosure, before connecting the adapter to the DNA fragment, the method further comprises a step of subjecting the DNA fragment to end repair and adenine nucleotide (A)-tailing.

In some embodiments of the present disclosure, primers used for PCR amplification comprise a common primer and a Barcode Primer.

In some embodiments of the present disclosure, the common primer has a sequence as shown in SEQ ID NO: 3.

In some embodiments of the present disclosure, the Barcode Primer has a sequence as shown in SEQ ID NO: 4.

In some embodiments of the present disclosure, the method further comprises a step of performing purification on the sequencing library, and the purification is performed by using magnetic beads.

In some embodiments of the present disclosure, the method further comprises a step of performing cyclization on the sequencing library to obtain a cyclization library.

In some embodiments of the present disclosure, the cyclization is performed using the following reaction system:

| Components | Volume | |
|---|---|---|
| 10× T4 DNA Ligase Buffer | 4-7 | μL |
| Splint Oligo (10 μM) | 4-7 | μL |
| T4 DNA Ligase (600 U/μL) | 0.5-2 | μL |
| Sequencing library | 35-45 | μL |

| | |
|---|---|
| ddH₂O | Make up 60 μL. |

In some embodiments of the present disclosure, the cyclization is performed by incubating the reaction system for the cyclization at 35-40 °C for 28-35 min.

In some embodiments of the present disclosure, the Splint Oligo has a sequence as shown in SEQ ID NO: 5.

In some embodiments of the present disclosure, the method further comprises a step of performing purification on the cyclization library, and the purification is performed by using magnetic beads.

According to some embodiments of the present disclosure, the present disclosure has at least the following beneficial effects:

According to the technical solution of the present disclosure, the adapter for multi-sample pooling library construction is provided with a primary tag (there are 48 Index tag sequences), and the PCR primer is provided with a secondary tag (there are 192 Barcode tag sequences provided by the MGI platform supplier, and further, with the increase of Barcode tags issued by the MGI platform supplier, the Barcode tag sequences will be increased accordingly, so that the number of samples for pooling can be further increased). By using two-level tags to label the samples in the library, there will be up to 9216 samples (48×192 combinations) that can be distinguished in each sequencing lane by the tags. Furthermore, the Index tag sequences are designed with base sequences of different lengths (4-10 bases) to optimize the base balance among the base sequences of different lengths, so that the adapter system can be used for library construction of various types of DNA fragments.

According to the technical solution of the present disclosure, two-level tags are designed for the sequencing library, so that the number of available adapters is greatly increased while effectively reducing the adapter synthesis cost, so as to provide enough adapters for large-scale sample pooling sequencing, thereby effectively solving the problem of insufficient adapters frequently occurring during multi-sample pooling sequencing. Moreover, unequal-length adapters having a 4-10 bp inner-layer tag sequence can be applied to the library construction of DNA fragments with identical gene sequence in a certain region, and can be used for but not limited to the construction of next-generation sequencing libraries for such as common PCR product sequencing, multiplex PCR product sequencing, gene-edited progeny large-scale sequencing identification, whole genome re-sequencing, low-depth re-sequencing, and simplified sequencing.

### BRIEF DESCRIPTION OF THE DRAWINGS

The present disclosure will be further described with reference to the accompanying drawings and the examples, wherein:

FIG. 1 is a schematic structural diagram of an adapter (SEQ ID NOS: 1-2) described in Example 1 of the present disclosure; and

FIG. 2 is a schematic structural diagram of a library (SEQ ID NOS: 246-247) described in Example 1 of the present disclosure.

### DETAILED DESCRIPTION

The concept and technical effects of the present disclosure will be described clearly and completely hereinafter with reference to examples, so as to fully understand the purpose, features and effects of the present disclosure. It is apparent that the described examples are only a part of the embodiments but not all the embodiments of the present disclosure. Based on the embodiments of the present invention, other embodiments obtained by those skilled in the art without inventive effort fall within the protection scope of the present disclosure.

### Example 1

In this example, a method for a two-level tags single-strand cyclization library based on MGI sequencing platform was provided, and the library was prepared as follows.
1. Experimental Subjects: total 288 DNA samples of rice population purity identification materials and rice blast resistant population selected single plant materials.
2. Adapter design and synthesis
   (1) the Following Sequences were Synthesized:
Sequence I: 5′-TTGTCTTCCTAAGGAACGACATGGCTACGATCCGACTTNNNNNNNNNNT-3′ (SEQ ID NO: 1), where the Ns at positions 39 to 48 represent the forward sequence of the Index tag, with the length of 4-10 bases;
Sequence II: 5′ Phos-NNNNNNNNNNAAGTCGGAGGCCAAGCGGTCTTAGGAAG ACAACTCACGAGTAGGCTAGA-3′ (SEQ ID NO: 2), where the Ns at positions 1 to 10 represent the reverse complementary sequence of the Index tag, with the length of 4-10 bases.

The Index tag sequences corresponding to each of the adapters are as shown in Table 1.

TABLE 1
48 Index tag sequences with unequal lengths

| Adapter No. | Index tag sequences |
|---|---|
| 1 | TACA (SEQ ID NO: 6) |
| 2 | ATGG (SEQ ID NO: 7) |
| 3 | GGTA (SEQ ID NO: 8) |
| 4 | CGAT (SEQ ID NO: 9) |
| 5 | ACTCA (SEQ ID NO: 10) |
| 6 | GACCA (SEQ ID NO: 11) |
| 7 | CTGTA (SEQ ID NO: 12) |
| 8 | AGTGCC (SEQ ID NO: 13) |
| 9 | TCAGGA (SEQ ID NO: 14) |
| 10 | CTCACGC (SEQ ID NO: 15) |
| 11 | GAACGCA (SEQ ID NO: 16) |
| 12 | TCGTCAGG (SEQ ID NO: 17) |
| 13 | ACGTACCA (SEQ ID NO: 18) |
| 14 | TGAACGC (SEQ ID NO: 19) |
| 15 | CTGT (SEQ ID NO: 20) |
| 16 | GACC (SEQ ID NO: 21) |
| 17 | ACTGG (SEQ ID NO: 22) |
| 18 | CTAGAG (SEQ ID NO: 23) |
| 19 | TGCA (SEQ ID NO: 24) |
| 20 | CATCG (SEQ ID NO: 25) |
| 21 | ACTACA (SEQ ID NO: 26) |
| 22 | GACTGC (SEQ ID NO: 27) |
| 23 | TGAGCAG (SEQ ID NO: 28) |
| 24 | GTGCAGAG (SEQ ID NO: 29) |
| 25 | CTAG (SEQ ID NO: 30) |
| 26 | GAGAC (SEQ ID NO: 31) |
| 27 | ACTCG (SEQ ID NO: 32) |
| 28 | TGCTCG (SEQ ID NO: 33) |
| 29 | GACGACGC (SEQ ID NO: 34) |
| 30 | ACTTGGC (SEQ ID NO: 35) |
| 31 | TGAA (SEQ ID NO: 36) |
| 32 | CTGC (SEQ ID NO: 37) |
| 33 | AGATG (SEQ ID NO: 38) |
| 34 | TACCGA (SEQ ID NO: 39) |
| 35 | CTGGA (SEQ ID NO: 40) |
| 36 | GCTACCA (SEQ ID NO: 41) |
| 37 | AGTCAAGCG (SEQ ID NO: 42) |
| 38 | CTGTCGCAC (SEQ ID NO: 43) |
| 39 | GACG (SEQ ID NO: 44) |
| 40 | TCAAGACCG (SEQ ID NO: 45) |
| 41 | AGCT (SEQ ID NO: 46) |
| 42 | CTACACGGAA (SEQ ID NO: 47) |
| 43 | TCTGGCAACG (SEQ ID NO: 48) |
| 44 | GAGACGACCA (SEQ ID NO: 49) |
| 45 | GCTA (SEQ ID NO: 50) |
| 46 | TGAGG (SEQ ID NO: 51) |
| 47 | ATCCAC (SEQ ID NO: 52) |
| 48 | CAGT (SEQ ID NO: 53) |

### (2) Adapter Preparation

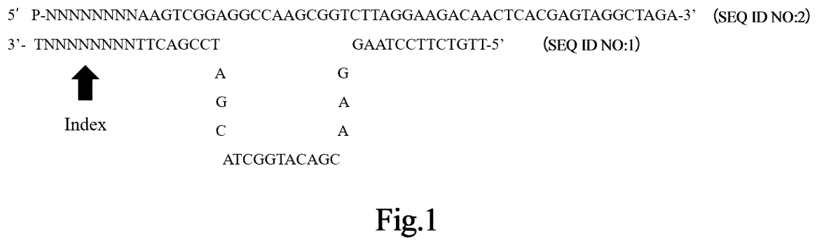

A DNA annealing buffer was added to the dry powders of Sequence I and Sequence II for dilution to obtain a solution of Sequence I with a concentration of 20 μM, and a solution of Sequence II with a concentration of 20 μM, respectively. The solution of Sequence I and the solution of Sequence II were mixed in an equal volume, and the obtained mixed solution was subjected to annealing on a PCR instrument to form the adapter. The annealing procedure involved: incubating at 95 °C for 3 min, and cooling by 1 °C every 90 sec until 25 °C, to obtain the adapter with a concentration of 10 μM. The adapter was temporarily stored at 4 °C or stored at −20 °C for long term. The adapter structure is shown in FIG. 1.

### 3. Cyclization Library Construction

### (1) Enzymatic Fragmentation of Genome

The genome was fragmented by restriction endonucleases TaqI and MspI. The system of double enzyme digestion is shown in Table 2, and the reaction procedure is shown in Table 3.

TABLE 2

| Components | Volume | |
|---|---|---|
| ddH₂O | 5 | μL |
| 10× T4 DNA Ligase Buffer | 3 | μL |
| TaqI (20 U/μL) | 1 | μL |
| MspI (20 U/μL) | 1 | μL |
| Genomic DNA (50 ng/μL) | 20 | μL |
| Total | 30 | μL |

TABLE 3

| Temperature | Period |
|---|---|
| 37 °C | 10 min |
| 65 °C | 10 min |
| 80 °C | 20 min |
| 4 °C | Hold |

After the digestion reaction, the genome was broken into DNA fragments with different lengths, and the fragments had 5′ protruding sticky ends CG at both terminals.

### (2) End Repair and A-Tailing

The reaction system for end repair and A-tailing was formulated according to Table 4.

TABLE 4

| Components | Volume | |
|---|---|---|
| Fragmented DNA | X | μL |
| End Repair/dA-Tailing Buffer | 6 | μL |
| End Repair/dA-Tailing Enzyme | 4 | μL |
| ddH₂O | Make up 60 μL | |
| Total | 60 | μL |

The reaction solution was gently blown or shaken with a pipette to mix, and centrifuged briefly to the bottom of the tube.

The PCR tube was placed into a PCR instrument, and the reaction procedure for end repair/dA-tailing reaction was set according to Table 5.

TABLE 5

| Temperature | Period |
|---|---|
| 105 °C for heating lid | On |
| 30 °C | 20 min |
| 72 °C | 20 min |
| 4 °C | Hold |

### (3) Adapter Ligation

Both ends of the above double enzyme digestion products were ligated with the adapters, such that each sample was labeled with a unique Index tag. The ligation system is shown in Table 6, and the ligation reaction procedure is shown in Table 7.

TABLE 6

| Components | Volume | |
|---|---|---|
| ddH₂O | 3 | μL |
| 10× T4 DNA Ligase Buffer | 1 | μL |
| Adapter (10 μM) | 5 | μL |
| T4 DNA Ligase (600 U/μL) | 1 | μL |
| Double enzyme digestion product | 30 | μL |
| Total | 40 | μL |

TABLE 7

| Temperature | Period |
|---|---|
| 23 °C | 60 min |
| 4 °C | Hold |

(4) Mixing Ligation Products into Sample Library

Every 24 ligation products with distinct Index tag sequences as a group were pooled into a sample library by combining an equal volume of 10 μL from each product. The ligation product library was purified using QIAquick PCR Purification Kit, and eluted and recovered using 20 μL 1×TE Buffer.

(5) Selecting DNA Fragments within Target Range

Using Pippin HT high-throughput automatic DNA fragment recovery system, DNA fragments within the range of 300-500 bp were selected and recovered from the ligation product library, and eluted and recovered with 30 μL TE Buffer. The concentration of the product recovered by PippinHT was detected using Qubit™ dsDNA HS Assay Kit, and TE Buffer was used to dilute the concentration to 1 ng/μL.

### (6) PCR Amplification

Using PCR common primer and 12 PCR Barcode primers (with the sequences as shown in Table 10 and Table 11, wherein the PCR common primer and the PCR Barcode primer were diluted with a DNA annealing buffer to a concentration of 10 μM), the DNA fragments within the target range were amplified through low-cycle-number PCR, such that each library was labeled with a unique Barcode tag. The PCR reaction system is shown in Table 8, and the reaction procedure is shown in Table 9.

TABLE 8

| Components | Volume | |
|---|---|---|
| ddH₂O | 13 | μL |
| KAPA HiFi HotStart ReadyMix | 25 | μL |
| PCR common primer (10 μM) | 1 | μL |
| PCR Barcode primer (10 μM) | 1 | μL |
| DNA fragments within target range | 10 | μL |
| Total | 50 | μL |

TABLE 9

| Temperature | Period | Cycle number |
|---|---|---|
| 95 °C | 3 min | 1 |
| 98 °C | 15 sec | 10 |
| 55 °C | 20 sec | |
| 72 °C | 30 sec | |
| 72 °C | 10 min | 1 |
| 4 °C | Hold | / |

TABLE 10

| Sequence name | Sequences (5′-3′) |
|---|---|
| PCR common primer | P-GAACGACATGGCTACGA |
| | (SEQ ID NO: 3) |
| Barcode primer | TGTGAGCCAAGGAGTTGNN |
| | NNNNNNNNTCTAGCCTACT |
| | CGTGAG (SEQ ID NO: 4) |
| Splint Oligo | GCCATGTCGTTCTGTGAG |
| | CCAAGG (SEQ ID NO: 5) |

Ns at positions 18 to 27 of the Barcode Primer sequence as shown in SEQ ID NO.4 is the sequence of the Barcode tag, and the Barcode tag can be any Barcode tags issued by the MGI platform supplier. At present, the MGI platform supplier provides 192 Barcode tag sequences (as shown in SEQ ID NO: 54 to SEQ ID NO: 245). With the increase of Barcode tags issued by the MGI platform supplier, new Barcode tag sequences can also be adopted, thus further increasing the number of samples for pooling.

The Barcode tag sequences in 12 PCR Barcode primers used in this example are shown in Table 11.

TABLE 11

| MGI Nos. of | |
|---|---|
| Barcode tags | Barcode tag sequences |
| 01 | GTGAGTGATG (SEQ ID NO: 54) |
| 02 | GAGTCAGCTG (SEQ ID NO: 55) |
| 03 | TGTCTGCGAA (SEQ ID NO: 56) |
| 04 | ATTGGTACAA (SEQ ID NO: 57) |
| 13 | AACCTAGATA (SEQ ID NO: 66) |
| 14 | TTGCCATCTC (SEQ ID NO: 67) |
| 15 | AGATCTTGCG (SEQ ID NO: 68) |
| 16 | CGCTATCGGC (SEQ ID NO: 69) |
| 41 | ATTCAACGGA (SEQ ID NO: 94) |
| 42 | AACTGTACTG (SEQ ID NO: 95) |
| 43 | GTACCTCAAT (SEQ ID NO: 96) |
| 44 | GACTTCTAAT (SEQ ID NO: 97) |

After the reaction, 50 μL of Ampure XP magnetic beads were added to purify and recover the PCR product, and 30 μL TE Buffer was used for elution. The concentration of the PCR purified product was detected using Qubit™ dsDNA HS Assay Kit, and TE Buffer was used to dilute the concentration to 5 ng/μL. 1 μL of the PCR purified product was taken for fragment range detection by Qsep100 automatic nucleic acid and protein analysis system.

### (7) Cyclization Library Preparation

12 PCR libraries with distinct Barcode tags were pooled by mixing 3 μL from each library into one tube, with a total DNA amount of 180 ng, and ddH₂O was added to make up 40 μL. The mixture was incubated in a PCR instrument at 95 °C for 3 min, and taken out for ice bath treatment to form a single-stranded separation product. The cyclization system was formulated according to Table 12, and the cyclization reaction procedure was performed according to Table 13.

TABLE 12

| Components | Volume | |
|---|---|---|
| ddH₂O | 8 | μL |
| 10× T4 DNA Ligase Buffer | 6 | μL |
| Splint Oligo as shown in SEQ ID NO: 5 (10 μM) | 5 | μL |
| T4 DNA Ligase (600 U/μL) | 1 | μL |
| Single-stranded separation product | 40 | μL |
| Total | 60 | μL |

TABLE 13

| Temperature | Period |
|---|---|
| 37 °C | 30 min |
| 4 °C | Hold |

After the cyclization reaction, the uncyclized, single-stranded and double-stranded DNAs were digested using DNA exonuclease. The exonuclease reaction system is shown in Table 14, and the reaction procedure is shown in Table 15.

TABLE 14

| Components | Volume | |
|---|---|---|
| ddH₂O | 7.5 | μL |
| 10× T4 DNA Ligase Buffer | 1 | μL |
| ExoI (20 U/μL) | 1 | μL |
| ExoIII (100 U/μL) | 0.5 | μL |
| Cyclization product | 60 | μL |
| Total | 70 | μL |

TABLE 15

| Temperature | Period |
|---|---|
| 37 °C | 30 min |
| 4 °C | Hold |

10 μL 0.1M EDTA (pH8.0) was added to quench the exonuclease activity. 1600 μL of Ampure XP magnetic beads were added to purify and recover the cyclization library, and 30 μL TE Buffer was used for elution. The concentration of the cyclization library was detected using Qubit™ ssDNA Assay Kit, and the cyclization library was stored at −20 °C until sequencing.

### (8) DNB Preparation, Loading and Sequencing

The obtained cyclization library was subject to DNB preparation according to the kit from MGI platform supplier.

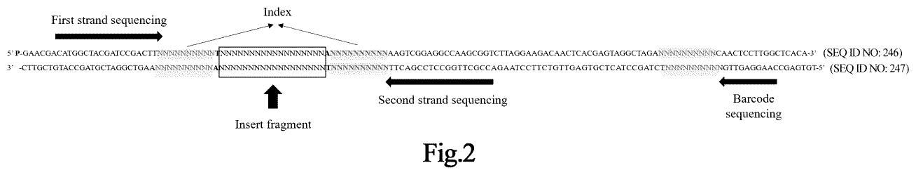

After DNB preparation, the product was loaded into MGISEQ-2000 or DNBSEQ-T7 platform for sequencing. The sequencing results showed that the total output data from 288 samples was 152 GB, and 91.39% of the average data reached the quality value Q30, showing good sequencing data quantity and quality. 90.99%, 96.5% and 89% of the data of the first, second and third bases at the starting position of the second strand sequencing reached the quality value Q30, respectively. After filtering and splitting the output data, the average data for each sample was 528 MB, of which the lowest data was 474 MB and the highest data was 586 MB. The schematic diagram of the library is shown in FIG. 2.

The embodiments of the present disclosure have been described in detail with reference to the accompanying drawings, but the present disclosure is not limited to the above embodiments, and various changes can be made within the knowledge of those ordinary skilled in the art without departing from the purpose of the present disclosure. In addition, the embodiments and the features in embodiments of the present disclosure can be combined with each other without conflict.

1. An adapter for multi-sample pooling library construction, comprising:
an adapter sense strand having a sequence as shown in SEQ ID NO: 1, and
an adapter anti-sense strand having a sequence as shown in SEQ ID NO: 2,
wherein the adapter sense strand and the adapter anti-sense strand form a double-stranded adapter after annealing.

2. The adapter according to claim 1, wherein, Ns at positions 39 to 48 of the sequence of the adapter sense strand represent a forward sequence of an Index tag, with a length ranging from 4 to 10 bases; and
Ns at positions 1 to 10 of the sequence of the adapter anti-sense strand represent a reverse complementary sequence of the Index tag, with a length ranging from 4 to 10 bases.

3. The adapter according to claim 2, wherein, the Index tag has a sequence selected from at least one of the group consisting of SEQ ID NO: 6 to SEQ ID NO: 53.

4. A kit for library construction based on MGI platform, comprising the adapter according to claim 1.

5. The kit according to claim 4, further comprising a common primer, and the common primer has a sequence as shown in SEQ ID NO: 3.

6. The kit according to claim 4, further comprising a Barcode Primer, and the Barcode Primer has a sequence as shown in SEQ ID NO: 4.

7. The kit according to claim 4, further comprising a Splint Oligo, and the Splint Oligo has a sequence as shown in SEQ ID NO: 5.

8. A method for next-generation sequencing library construction, comprising the following steps:
connecting the adapter according to claim 1 to a DNA fragment to obtain a DNA fragment with the adapter; and
performing PCR amplification on the DNA fragment with the adapter to obtain a sequencing library.

9. The method according to claim 8, further comprising, before connecting the adapter to the DNA fragment, annealing two sequences of the adapter.

10. The method according to claim 8, further comprising a step of performing cyclization on the sequencing library.

11. The kit according to claim 6, wherein Ns at positions 18 to 27 of the sequence of the Barcode Primer as shown in SEQ ID NO: 4 represent a sequence of a Barcode tag.

12. The method according to claim 8, wherein the DNA fragment is provided by ultrasonic interruption, fragmentation enzyme digestion, or restriction enzyme digestion.

13. The method according to claim 9, wherein the annealing is performed by incubating at 92-98 °C for 2-5 min, and cooling by 0.8-1.2 °C per 85-95 sec until 20-28 °C.

14. The method according to claim 9, wherein in the annealing, a mass ratio of the two sequences is 1:1.

15. The method according to claim 8, wherein before connecting the adapter to the DNA fragment, the method further comprises a step of subjecting the DNA fragment to end repair and adenine nucleotide (A)-tailing.

16. The method according to claim 8, wherein primers used for PCR amplification comprise a common primer and a Barcode Primer.

17. The method according to claim 16, wherein the common primer has a sequence as shown in SEQ ID NO: 3.

18. The method according to claim 16, wherein the Barcode Primer has a sequence as shown in SEQ ID NO: 4.

19. The method according to claim 8, wherein the method further comprises a step of performing purification on the sequencing library, and the purification is performed by using magnetic beads.

20. The method according to claim 10, wherein the cyclization is performed by incubating a reaction system comprising:

| Components | Volume | |
|---|---|---|
| 10× T4 DNA Ligase Buffer | 4-7 | μL |
| Splint Oligo (10 μM) | 4-7 | μL |
| T4 DNA Ligase (600 U/μL) | 0.5-2 | μL |
| Sequencing library | 35-45 | μL |
| | |
|---|---|
| ddH₂O | Make up 60 μL. |

for the cyclization at 35-40 °C for 28-35 min.
